(19) United States
(12) Patent Application Publication
(10) Pub. No.: US 2026/0131135 A1
(43) Pub. Date: May 14, 2026

(54) HERMETIC DRIVELINE FOR IMPLANTABLE MEDICAL DEVICES

(71) Applicant: Procyrion, Inc., Houston, TX (US)

(72) Inventors: Christopher A. Durst, Houston, TX (US); Ronald G. Earles, Houston, TX (US); Omar M. Benavides, Houston, TX (US); Av Edidin, Houston, TX (US)

(21) Appl. No.: 19/383,414

(22) Filed: Nov. 7, 2025

Related U.S. Application Data
(60) Provisional application No. 63/718,507, filed on Nov. 8, 2024.
(60) Provisional application No. 63/814,254, filed on May 29, 2025.

(51) Int. Cl. A61M 60/878; A61M 60/13; A61M 60/226; A61M 60/422; A61M 60/806; H01R 24/58

(52) U.S. Cl. CPC A61M 60/878; A61M 60/13; A61M 60/226; A61M 60/422; A61M 60/806; H01R 24/58; H01R 2201/12

(57) ABSTRACT

A power lead is provided that includes an elongate body, a feedthrough body, and a housing enclosing the feedthrough body. The elongate body has a proximal end and a distal end and is configured to connect to a therapy device adjacent to the distal end and to a controller adjacent to the proximal end. The elongate body has a plurality of lumens, each one of which houses a length of a conductor of a plurality of conductors. The feedthrough body has a plurality of channels to support a connector pin electrically connected to a conductor of the plurality of conductors. A distal portion of the connector pin is disposed in a recess extending between an opening of the housing and a distal end of the feedthrough body.

### INCORPORATION BY REFERENCE TO ANY PRIORITY APPLICATIONS

This application claims priority to U.S. patent application Ser. No. 63/718,507, filed on Nov. 8, 2024, and to U.S. patent application Ser. No. 63/814,254, filed on May 29, 2025, the entire contents of each of which are incorporated by reference herein in their entirety and for all purposes. Any and all applications for which a foreign or domestic priority claim is identified in the Application Data Sheet as filed with the present application are hereby incorporated by reference under 37 CFR 1.57.

### BACKGROUND

### Field

The field relates to hermetic provision of electrical or motive power through a driveline to a therapeutic implant.

### Description of the Related Art

In the field of cardiac assist devices and mechanical circulatory support, blood pumps are used to support the heart in circulating blood through the body. Some of these blood pumps are intravascular blood pumps and are designed or adapted for use within blood vessels.

### SUMMARY

In one embodiment, a power lead is provided that includes an elongate body, a feedthrough body, and a housing. The elongate body has a proximal end and a distal end and is configured to connect to a medical device (e.g., a therapy device, a percutaneous medical device, such as a blood pump) adjacent to the distal end and to a controller adjacent to the proximal end. The elongate body can electrically connect the medical device (e.g., a therapy device, a percutaneous medical device, such as a blood pump) to the controller in some embodiments. The elongate body can physically connect the medical device (e.g., a therapy device, a percutaneous medical device, such as a blood pump) to the controller in some embodiments. The elongate body can physically and electrically connect the medical device (e.g., a therapy device, a percutaneous medical device, such as a blood pump) to the controller in some embodiments. The elongate body has one or more, e.g., a plurality of, lumen(s). One lumen can be for housing a rotatable drive shaft. The one lumen for housing the drive shaft can be a single lumen. The one lumen for housing the drive shaft can be a central lumen. In some cases, each lumen can be configured to house a portion of a length of a conductor of a plurality of conductors. The feedthrough body has a proximal end and a distal end. The feedthrough body has a plurality of channels. Each channel is configured to house a proximal portion of a connector pin configured to be electrically connected to a conductor of the plurality of conductors. The housing encloses the feedthrough body. The housing having a length greater than the length of the feedthrough body such that a distal portion of the connector pin is disposed in (e.g., cantilevered in) a blind recess extending between an opening of the housing and the distal end of the feedthrough body.

In further variations of the foregoing, the housing can enclose a plurality of sockets configured to receive connector pins disposed on the medical device (e.g., a therapy device, a percutaneous medical device, such as a blood pump), which can be an intravascular device, e.g., a percutaneous blood pump.

In another embodiment, a drive unit is provided that includes a motor housing and a connection portion. The motor housing can enclose a motor configured to be activated to produce rotational motion. The connection portion can comprise a coupling surface and an electrical circuit. The electrical circuit can be electrically connected to a plurality of pin receptacles, each pin receptacle configured to receive a pin of a plurality of pins of a power lead to electrically connect the motor to a controller. In one variation, the electrical circuit can be electrically connected to a plurality of pins, each pin being configured to be inserted into a plurality of pin receptacles of a power lead to electrically connect the motor to a controller.

In another embodiment, a method of assembling a medical device (e.g., a therapy device, a percutaneous medical device, such as a blood pump) is provided. A connection portion of an implantable electrical device (e.g., a therapy device, an implantable electrical device, such as a percutaneous blood pump or other intravascular electrical device) is advanced into a blind recess of a distal end of a power lead. A plurality of pins disposed on one of the implantable electrical device and the power lead is engaged with a plurality of sockets disposed on the other of the implantable electrical device and the power lead. Electrical connections are thereby made between the power lead and the medical device (e.g., a therapy device, a percutaneous medical device, such as a blood pump). A seal of the power lead and/or the implantable electrical device from fluid ingress in and around the blind recess is provided.

In one embodiment, a power lead comprises: an elongate body having a proximal end and a distal end and being configured to electrically and mechanically connect to a therapy device adjacent to the distal end and to a controller adjacent to the proximal end, the elongate body comprising a plurality of lumens, each lumen configured to house a portion of a length of a conductor of a plurality of conductors; a feedthrough body having a proximal end and a distal end and comprising a plurality of channels, each channel configured to house a proximal portion of a connector pin configured to be electrically connected to a conductor of the plurality of conductors; and a housing enclosing the feedthrough body, the housing having a length greater than the length of the feedthrough body such that a distal portion of the connector pin is disposed in a blind recess extending between an opening of the housing and the distal end of the feedthrough body.

In some embodiments, the feedthrough body comprises a ceramic material. In some embodiments, the housing comprises an inert metal. In some embodiments, the housing comprises titanium. In some embodiments, a welding feature is disposed between the proximal end of the feedthrough body and the elongate body, the welding feature comprise a material configured to enhance a weld purchase to the housing. In some embodiments, the feedthrough body comprises a sealing passage configured to convey a sealant into cavities in and/or around the feedthrough body. In some embodiments, a sealant is disposed in interstices of the power lead in and around the feedthrough body. In some embodiments, the housing comprises openings at each of an enlarged distal end and a tapered proximal end, the opening at the distal end configured to receive a feedthrough body assembly, a proximal end of the feedthrough body assembly configured to protrude through the opening at the proximal end of the housing. In some embodiments, the feedthrough body assembly comprises a weld purchase component at a proximal end of the feedthrough body, the weld purchase component configured to enhance a weld between the feedthrough body assembly and the housing. In some embodiments, an alignment key to facilitate automatic alignment with a portion of the therapy device. In some embodiments, the alignment key extends parallel to the alignment pin. In some embodiments, the alignment key is longer than the connector pin.

In some embodiments, an implantable device includes the aforementioned power lead and a therapy device comprising an electrical circuit electrically connected to a plurality of sockets, each socket of the plurality of sockets configured to receive one of the connector pins of the plurality of connector pins to electrically connect the therapy device to the controller. In some embodiments, the therapy device comprises a pump. In some embodiments, a weld formed between a distal end of the housing of the power lead and a surface of the therapy device adjacent to the plurality of sockets. In some embodiments, the therapy device and the power lead are joined in a manner preventing leaking of helium in a helium leak test. In some embodiments, the feedthrough body comprises a disk disposed in the housing, a distal portion of the disk defining a portion of the blind recess, a proximal portion of the disk disposed distal to a volume through which each of the conductors of the plurality of conductors can be routed. In some embodiments, an enclosure is disposed between the proximal end of the disk and the distal end of the elongate body, the enclosure configured to at least partially surround the volume through which each of the conductors of the plurality of conductors can be routed. In some embodiments, the enclosure comprises a polymeric body. In some embodiments, the enclosure comprises a groove configured to receive a seal member. In some embodiments, the seal member comprises an O-ring. In some embodiments, the enclosure is disposed within the housing. In some embodiments, an adhesive is disposed between an outer surface of the enclosure and an inner surface of the housing. In some embodiments, an adhesive is disposed within the enclosure to control fluid ingress into the elongate body. In some embodiments, the volume is at least partially occupied by an adhesive to control fluid ingress into the elongate body. In some embodiments, a conductor passage assembly comprises a support element and a plurality of tubular bodies, each tubular body configured to receive a distal portion of a conductor of the plurality of conductors. In some embodiments, the support element comprises a planar portion disposed proximally of the feedthrough body and a flange extending proximally from the planar portion between two of the tubular bodies of the plurality of tubular bodies. In some embodiments, each adjacent pair of tubular bodies is separated by a flange of the support element.

In another embodiment, a drive unit comprises: a motor housing enclosing a motor configured to be activated to produce rotational motion; and a connection portion comprising a coupling surface and an electrical circuit, the electrical circuit being electrically connected to a plurality of pin receptacles, each pin receptacle configured to receive a pin of a plurality of pins of a power lead to electrically connect the motor to a controller.

In some embodiments, the coupling surface is configured to facilitate a sealing connection to the power lead. In some embodiments, the sealing connection can comprise a circumferential weld. In some embodiments, the pin receptable is configured to electrically connect to a pin of the power lead via a press-fit connection. In some embodiments, a pin guide is disposed over the connection portion. In some embodiments, the pin guide comprises a base member and one or more fingers extending non-parallel relative to an outer periphery of the base member, the one or more fingers extending along a channel of the connection portion. In some embodiments, the pin guide comprises a plurality of notches, each notch disposed between adjacent fingers.

In another embodiment, a method of assembling a therapy device includes: advancing a connection portion of an implantable electrical device into a blind recess of a distal end of a power lead; engaging a plurality of pins disposed on one of the implantable electrical device and the power lead with a plurality of sockets disposed on the other of the implantable electrical device and the power lead, such that electrical connections are made between the power lead and the implantable electrical device; and providing a seal of the power lead and/or the implantable electrical device from fluid ingress in and around the blind recess. In some embodiments, a connection portion of a distal end of a power lead can be advanced into a blind recess of an implantable electrical device, or vice versa. In some embodiments, only one pin and one socket may be used.

In some embodiments, providing the seal comprises filling interstices of the power lead with a flowable curable medium: In some embodiments, providing the seal comprises providing a weld at or adjacent to a periphery on the blind recess. In some embodiments, the implantable electrical device comprises a motor configured to generate rotation of an output shaft. In some embodiments, the implantable electrical device comprises a blood pump and providing the seal comprises limiting ingress of blood into the power lead. In some embodiments, engaging the plurality of pins disposed on one of the implantable electrical device and the power lead with a plurality of sockets disposed on the other of the implantable electrical device and the power lead comprises inserting pins into a press-fit receptacle. In some embodiments, the press-fit receptacle provide an electrical connection between the pin and the receptacle without the need for solder.

In another embodiments, an implantable medical device includes: a first connector pin configured to electrically connect to a controller; a therapy device comprising an electrical component connected to a first socket at an end portion of the electrical component, the first socket receiving the first connector pin; and a pin guide connected to the end portion of the electrical component, the pin guide comprising a first pin hole aligned with the first socket, such that the first connector pin extends through the first pin hole to connect to the first socket.

In some embodiments, the pin guide comprises a base member and one or more fingers extending non-parallel relative to an outer periphery of the base member, the one or more fingers extending along a channel of the end portion of the electrical component. In some embodiments, a plurality of pin holes includes the first pin hole. In some embodiments, the pin guide comprises one or more notches in a rim or outer periphery of the base member. In some embodiments, the electrical component comprises a motor housing and the end portion comprises a drive cap. In some embodiments, a circuit board ia connected to the drive cap, the circuit board comprising the first socket. In some embodiments, a motor wire is electrically connected to the first socket by way of a solder connection. In some embodiments, a first notch of the one or more notches at least partially circumferentially overlaps the solder connection. In some embodiments, the pin guide comprises a relief surface to accommodate the solder connection and motor wire. In some embodiments, a plurality of notches includes the first notch, wherein each notch of the plurality of notches is disposed circumferentially between adjacent fingers extending non-parallel from a base member of the pin guide. In some embodiments, the first pin hole has a first width at a proximal surface and a second width at a distal surface, the second width smaller than the first width. In some embodiments, the first pin hole comprises a first tapered sidewall and a second vertical sidewall extending from the first tapered sidewall. In some embodiments, the therapy device comprises a pump. In some embodiments, a feedthrough body is connected to the first connector pin, the feedthrough body disposed proximal the pin guide. In some embodiments, a housing is disposed about the feedthrough body and the pin guide.

In another embodiments, a pin guide includes: a base member; a plurality of fingers extending non-parallel relative to an outer periphery of the base member; and a first pin hole extending through the base member.

In some embodiments, a first notch is disposed in a rim of the pin guide or an outer periphery of the base member, the first notch disposed between two circumferentially-adjacent fingers. In some embodiments, a plurality of notches includes the first notch. In some embodiments, the first pin hole has a first width at a proximal surface and a second width at a distal surface, the second width smaller than the first width. In some embodiments, the first pin hole comprises a first tapered sidewall and a second vertical sidewall extending from the first tapered sidewall. In some embodiments, a relief surface can be disposed on a distal surface of the base member.

In another embodiments, a support collar includes: a lower cavity; an upper cavity having an inner surface shaped to conform to an electrical component, the inner surface including a plurality of projections sized and shaped to mate with corresponding notches of the electrical component; and a shelf separating the upper and lower cavities, the lower cavity being wider than the upper cavity along the entire periphery of the lower cavity.

In some embodiments, the plurality of projections are curved.

In another embodiments, a compression tool includes: a central opening; an arm extending across the central opening; and a tip extending longitudinally from the art into the central opening.

In some embodiments, the arm is integrally formed with an upper surface of an annular base.

In another embodiments, a method of assembling a portion of a medical device includes: disposing a support collar over a drive cap; inserting a circuit board into a cavity of the support collar over the drive cap; and dispensing an adhesive into the cavity to adhere the circuit board to the drive cap.

In some embodiments, the method includes disposing a compression tool over the support collar and pressing the compression tool longitudinally against the circuit board. In some embodiments, the compression tool comprises a tip disposed over a central opening, the method comprising pressing the tip against the circuit board to support the circuit board while the adhesive is being dispensed In some embodiments, the method comprises pressing the tip against the circuit board to support the circuit board while the adhesive is being cured.

### BRIEF DESCRIPTION OF THE DRAWINGS

These and other features, aspects and advantages are described below with reference to the drawings, which are intended for illustrative purposes and should in no way be interpreted as limiting the scope of the embodiments. Furthermore, various features of different disclosed embodiments can be combined to form additional embodiments, which are part of this disclosure. In the drawings, like reference characters denote corresponding features consistently throughout similar embodiments. The following is a brief description of each of the drawings.

FIG. 1A is a schematic perspective, partially-exploded view of a blood flow assist system, according to various embodiments.

FIG. 1B is a schematic perspective view of a pump at a distal portion of the blood flow assist system of FIG. 1A.

FIG. 1C is a schematic perspective, partially-exploded view of the pump of FIG. 1B.

FIG. 1D is a schematic side sectional view of a motor housing according to various embodiments.

FIG. 1E is a schematic perspective view of a motor and a motor mount support.

FIG. 1F is a schematic perspective view of a distal end of a power lead having lumens shaped to receive conductors that are configured to supply power to the motor.

FIG. 1G is a schematic perspective view of a proximal end portion of the power lead.

FIG. 1H is a schematic side view of the pump disposed in a collapsed configuration in a delivery sheath.

FIG. 1I is a schematic perspective view of a retrieval feature used to remove the pump, according to some embodiments.

FIG. 1J is a cross-sectional view of an alternative embodiment in which a drive shaft is coupled to a motor configured to be disposed outside the patient when the pump is in use.

FIGS. 2A-2E illustrate an example embodiment of a housing 229 for an electromechanical device.

FIGS. 3A-3B illustrate additional embodiments of a feedthrough assembly for an electromechanical device (e.g., a heart pump).

FIGS. 4A-4D illustrate an embodiment of a feedthrough body including an alignment key to facilitate automatic alignment with the remainder of the pump.

FIGS. 5A-5K schematically illustrate various embodiments of a pin guide for a feedthrough assembly.

FIGS. 6A-6H schematically illustrate various components for assembling a portion of a feedthrough assembly.

### DETAILED DESCRIPTION

Refer now to the drawings wherein depicted elements are not necessarily shown to scale and wherein like or similar elements are designated by the same reference numeral through the several views.

Referring to the drawings in general, it will be understood that the illustrations are for the purpose of describing particular implementations of the disclosure and are not intended to be limiting thereto. While most of the terms used herein will be recognizable to those of ordinary skill in the art, it should be understood that when not explicitly defined, terms should be interpreted as adopting a meaning presently accepted by those of ordinary skill in the art.

### I. OVERVIEW OF BLOOD FLOW ASSIST SYSTEMS

Various embodiments disclosed herein relate to a blood flow assist system 1

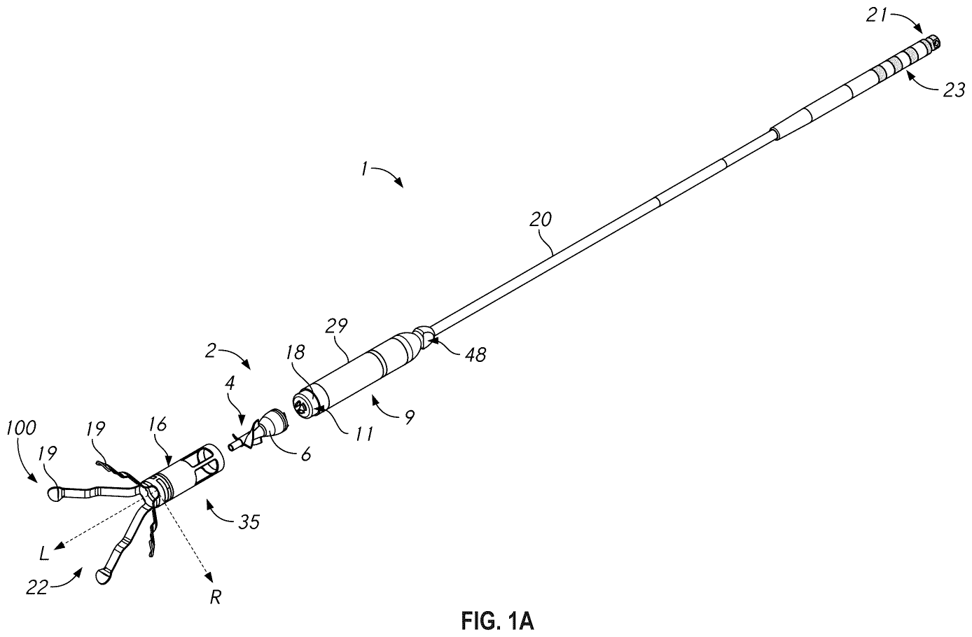

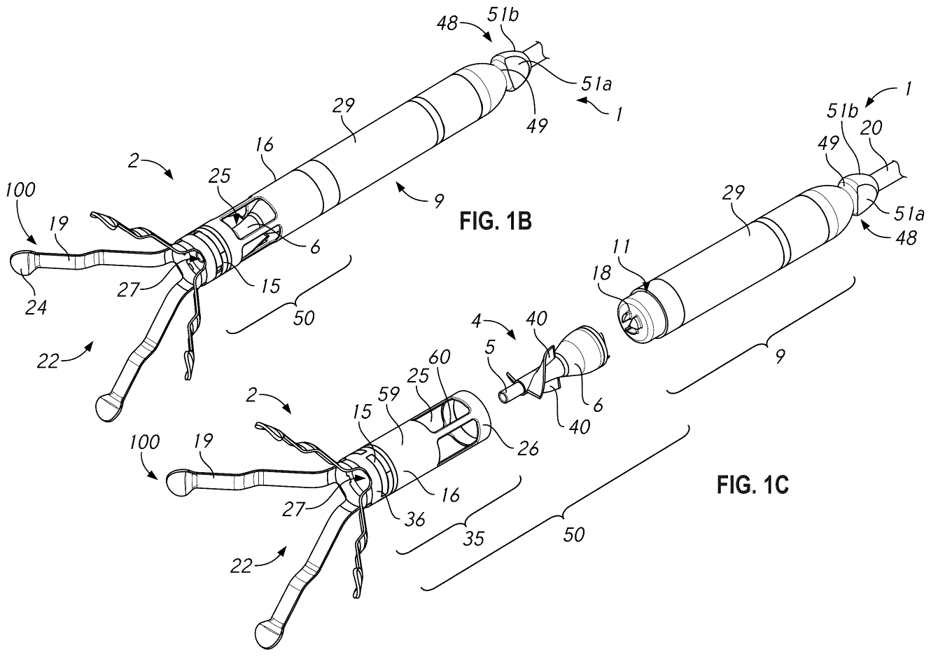

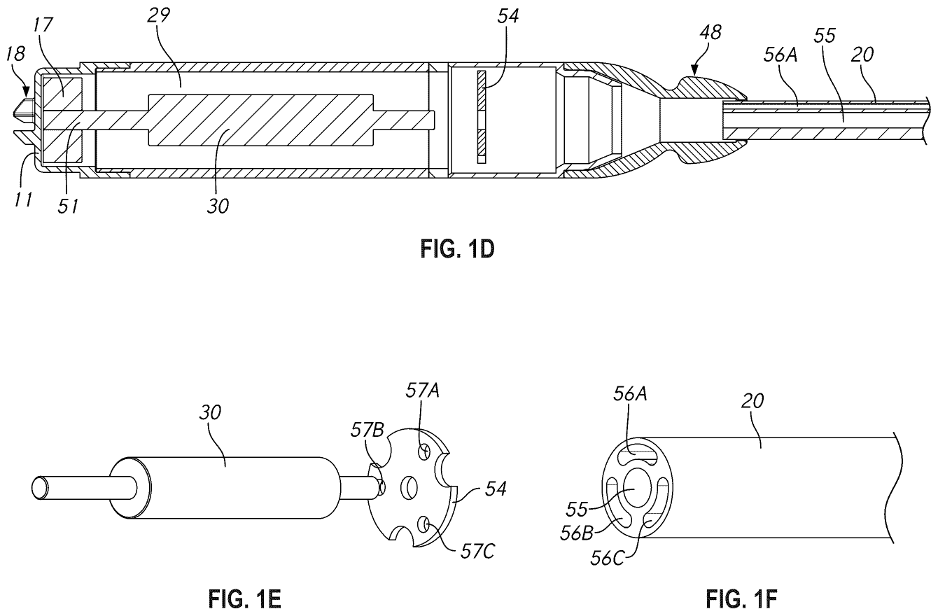

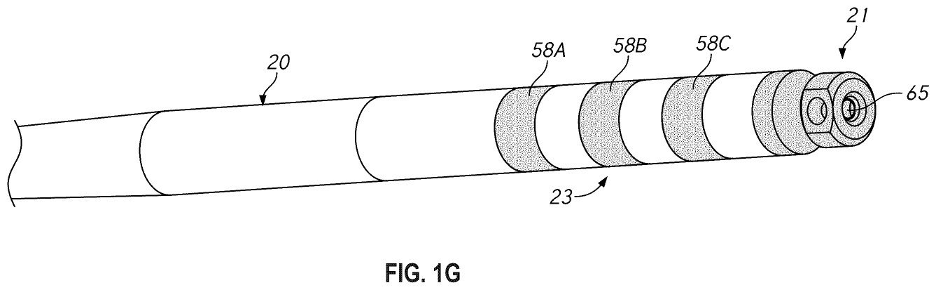

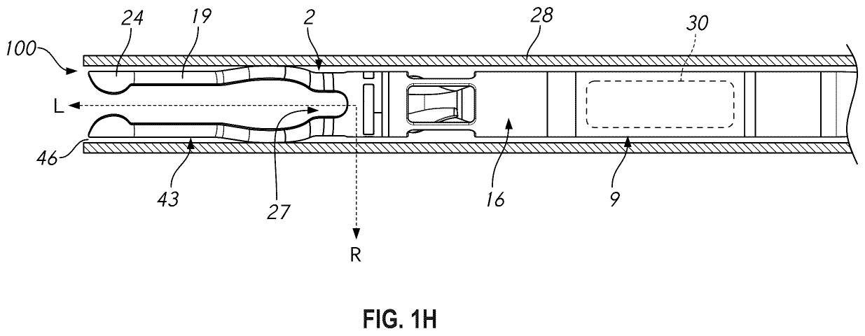

configured to provide circulatory support to a patient, as illustrated in FIGS. 1A-1I. The system 1 can be sized for intravascular delivery to a treatment location within the circulatory system of the patient, e.g., to a location within the descending aorta of the patient. As shown in FIG. 1A, the system 1 can have a proximal end 21 with a connector 23 configured to connect to an external control system, e.g., a console (not shown). The connector 23 can provide electrical communication between the control system and a power lead 20 extending distally along a longitudinal axis L from the connector 23 and the proximal end 21. The power lead 20 can comprise an elongate body that electrically and mechanically connects to a pump 2 at or near a distal end 22 of the blood flow assist system 1, with the distal end 22 spaced apart from the proximal end 21 along the longitudinal axis L. As explained herein, the power lead 20 can also serve as a flexible tether configured to oppose loads applied in opposite directions at opposite ends of the power lead 20.

The pump 2 can comprise a pump head 50 including a pump housing 35 connected to a drive unit 9 that includes a motor housing 29. A retrieval feature 48 can be provided at a proximal end portion of the pump 2. In some embodiments, the retrieval feature can be coupled with the distal end of the power lead 20 between the power lead 20 and the motor housing 29. After a procedure, the clinician can remove the pump 2 from the patient by engaging a tool (e.g., a snare, a clamp, hook, etc.) with the retrieval feature 48 to pull the pump 2 from the patient. For example, the retrieval feature 48 can comprise a neck 49 (e.g., a reduced diameter section) at a proximal curved portion 51c of the motor housing 29 and an enlarged diameter section disposed proximal the neck 49. The enlarged diameter section can comprise a first curved portion 51a and a second curved portion 51b, as shown in FIGS. 1B, 1C, and 1I. The first and second curved portions 51a, 51b can comprise convex surfaces, e.g., convex ball portions. The first and second curved portions 51a, 51b can have different radii of curvature. For example, as shown in FIG. 1I, the first curved portion 51a can have a larger radius of curvature than the second curved portion 51b. The first curved portion 51a can be disposed on opposing sides of the retrieval feature 48 in some embodiments. The second curved portion 51b can be disposed around the first curved portion 51a and can have a radially-outward facing surface and a proximally-facing convex surface coupled to the distal end of the power lead 20. The neck 49 can have a first depth at a first circumferential position of the retrieval feature 48 and a second depth less than the first depth at a second circumferential position of the retrieval feature 48 spaced apart from the first circumferential position.

Beneficially, as shown in FIG. 1I, one or more first planes P1 extending parallel to the longitudinal axis L and intersecting the first curved portion 51a can have a first angle or taper between the proximal curved portion 51c of the motor housing 29 and the first curved portion 51a. One or more second planes P2 extending parallel to the longitudinal axis L and intersecting the second curved portion 51b can have a second angle or taper (which is different from the first angle or taper) between the proximal curved portion 51c of the motor housing 29 and the second curved portion 51b. The first angle or taper can provide a gradual, continuous (generally monotonically decreasing) geometric transition between the proximal curved portion 51c of the motor housing 29 and the power lead 20, which can provide for smooth blood flow and reduce the risk of thrombosis. The second curved portion 51b can serve as a lobe that extends radially outward, e.g., radially farther out than the first curved portion 51a. The second curved portion 51b can be used to engage with a retrieval device or snare to remove the pump 2 from the anatomy. Some cross sections through the longitudinal axis of the retrieval feature 48 can contain a substantial neck (e.g., a local minimum in the radius of curvature measured along its central axis) while other cross sections through the longitudinal axis of the retrieval feature 48 can contain an insubstantial local minimum or no local minimum. In the illustrated embodiment, there are two first curved portions 51a that can serve as a dual lobe retrieval feature. In other embodiments, more or fewer lobes can be provided to enable pump retrieval while ensuring smooth flow transitions between the motor housing 29 and power lead 20.

As shown in FIGS. 1B-1C, 1E, and 1I, the neck 49 can be disposed between the curved portions 51a, 51b and a proximally-facing convex surface 51c of the motor housing 29. In the illustrated embodiment, the retrieval feature 48 can be coupled to or integrally formed with the motor housing 29. In other arrangements, the retrieval feature 48 can be disposed at other locations of the pump 2. As shown, the retrieval feature 48 can be symmetrical and continuously disposed about the longitudinal axis L. In other arrangements, the retrieval feature 48 can comprise a plurality of discrete surfaces spaced apart circumferentially and/or longitudinally. In the illustrated embodiments, the motor housing 29 (and motor) can be part of the pump 2 and disposed inside the vasculature of the patient in use. In other embodiments, however, the motor housing 29 (and motor) can be disposed outside the patient and a drive cable can connect to the impeller 6.

As shown in FIGS. 1A-1C, the drive unit 9 can be configured to impart rotation to an impeller assembly 4 disposed in the pump housing 35 of the pump head 50. As explained herein, the drive unit 9 can include a drive magnet 17 (see FIG. 1D) and a motor 30 (see FIGS. 1D-1E) disposed in the motor housing 29 capped by a distal drive unit cover 11. The motor 30 is shown schematically in FIG. 1D. The drive unit cover 11 can be formed with or coupled to a drive bearing 18. The drive magnet 17 can magnetically couple with a corresponding driven or rotor magnet (not shown) of the impeller assembly 4 that is disposed proximal the impeller 6 within the shroud 16. The power lead 20 can extend from the treatment location to outside the body of the patient, and can provide electrical power (e.g., electrical current) and/or control to the motor 30. Accordingly, no spinning drive shaft extends outside the body of the patient in some embodiments. As explained herein, the power lead 20 can energize the motor 30, which can cause the drive magnet 17 to rotate about the longitudinal axis L, which can serve as or be aligned with or correspond to an axis of rotation. Rotation of the drive magnet 17 can impart rotation of the rotor magnet and a primary or first impeller 6 of the impeller assembly 4 about the longitudinal axis L. For example, as explained herein, the rotor magnet (which can be mechanically secure to an impeller shaft 5) can cause the impeller shaft 5 (which can serve as a flow tube) and the first impeller 6 to rotate to pump blood. In other embodiments, the drive unit 9 can comprise a stator or other stationary magnetic device. The stator or other magnetic device can be energized, e.g., with alternating current, to impart rotation to the rotor magnet. In the illustrated embodiments, the impeller 6 can have one or a plurality of blades 40 extending radially outward along a radial axis R that is radially transverse to the longitudinal axis L. For example, the first impeller 6 can have a plurality of (e.g., two) longitudinally-aligned blades 40 that extend radially outwardly from a common hub and that have a common length along the longitudinal axis L. The curvature and/or overall profile can be selected so as to improve flow rate and reduce shear stresses. Skilled artisans would appreciate that other designs for the first impeller 5 may be suitable.

As shown in FIGS. 1A-1C, the impeller assembly 4 can be disposed in a shroud 16. The impeller shaft 5 can be supported at a distal end by a sleeve bearing 15 connected to a distal portion of the shroud 16. A support structure such as a localization system 100 (discussed further below) can comprise a base portion 36 coupled with the sleeve bearing 15 and/or the shroud 16. In some embodiments, the base portion 36, the sleeve bearing 15, and/or the shroud 16 can be welded together. In other embodiments, the sleeve bearing 15 and/or the shroud 16 can be formed as one part. The base portion 36 of the support structure or localization system 100 (which can be part of or serve as a support structure), the sleeve bearing 15, and the shroud 16 can cooperate to at least partially define the pump housing 35, as shown in FIGS. 1A and 1C. The localization system 100 can comprise a plurality of self-expanding struts 19 having convex contact pads 24 configured to contact a blood vessel wall to maintain spacing of the pump housing 35 from the wall of the blood vessel in which the pump housing 35 is disposed. In FIGS. 1A-1C, the struts 19 of the localization system 100 are illustrated in an expanded, deployed configuration, in which the contact pads 24 extend radially outward to a position in which the contact pads 24 would contact a wall of a blood vessel within which the pump 2 is disposed to at least partially control position and/or orientation of the pump head 50 relative to the blood vessel wall, e.g., to anchor, the pump 2 during operation of the system 1.

A first fluid port 27 can be provided distal the impeller assembly 4 at a distal end of the pump housing 35. The shroud 16 can comprise a proximal ring 26 coupled with the motor housing 29 and a plurality of second fluid ports 25 formed in a proximal portion of the shroud 16 adjacent (e.g., immediately distal) the proximal ring 26. As shown in FIG. 1C, the second fluid ports 25 can comprise openings formed between axially-extending members 60 (also referred to as pillars) that extend along the longitudinal axis L (which may also serve as a longitudinal axis of the pump head 2 and/or pump housing 35) between the proximal ring 26 and a cylindrical section 59 of the shroud 16. In some embodiments, the axially-extending members 60 can be shaped or otherwise be configured to serve as vanes that can shape or direct the flow of blood through the second fluid ports 25. For example, in various embodiments, the axially-extending members 60 can be angled, tapered, or curved (e.g., in a helical pattern) to match the profile of the impeller blades 40 and/or to accelerate blood flow through the pump 2. In other embodiments, the axially-extending members 60 may not be angled to match the blades 40. In some embodiments, the first fluid port 27 can comprise an inlet port into which blood flows. In such embodiments, the impeller assembly 4 can draw blood into the first fluid port 27 and can expel the blood out of the pump 2 through the second fluid ports 25, which can serve as outlet ports. In other embodiments, however, the direction of blood flow may be reversed, in which case the second fluid ports 25 may serve as fluid inlets and the first fluid port 27 may serve as a fluid outlet.

As shown in FIGS. 1A-1D, the system 1 comprises the drive unit 9 with the motor 30 that can be sealed in the motor housing 29. The drive magnet 17 can be rotatable by the motor 30 by way of a motor shaft 51. The motor 30 can electrically connect to the power lead 20. The power lead 20 can serve as a flexible tether that comprises an elongate tension member configured to oppose loads applied in opposite directs at opposite ends of the power lead 20. In one embodiment the power lead 20 is hollow, as discussed further below. As shown in FIGS. 1D and 1F, the power lead 20 can comprise an insulating body having a central lumen 55 and a plurality of (e.g., three) outer lumens 56A-56C extending along a length of the power lead 20. One or more electrical conductors can be disposed in the hollow elongate power lead 20 and can be configured to convey current to the motor 30 from a source, such as the external control system. For example, in some embodiments, the outer lumens 56A-56C can be sized and shaped to receive corresponding electrodes or electrical wires (not shown) to provide electrical power to the motor 30. For example, the lumens 56A-56C can receive, wires configured to supply ground and drive voltage to corresponding windings on the motor. The electrodes can extend through corresponding openings 57A-57C of a motor mounting support 54 configured to support the motor 30. The central lumen 55 can be sized and shaped to receive an elongate stiffening member or guidewire (not shown). The stiffening member or guidewire can be inserted through an opening 65 at the proximal end 21 (see FIG. 1G) into the central lumen 55 during delivery to help guide the pump 2 to the treatment location or maintain the pump 2 in a given location. The stiffening member or guidewire can be easily inserted and removed when finished. As shown in FIG. 1G, the connector 23 near the proximal end 21 of the system 1 can have electrical contacts 58A-58C electrically connected to the wires or conductors in the corresponding outer lumens 56A-56C. The contacts 58A-58C can comprise rings spaced apart by an insulating material and can be configured to electrically connect to corresponding electrical components in the control system or console (not shown).

Beneficially, the blood flow assist system 1 can be delivered percutaneously to a treatment location in the patient. FIG. 1H shows the pump 2 disposed within an elongate sheath 28. As shown, the struts 19 are held in a collapsed configuration by the inner wall of the sheath 28. As discussed further below, the struts 19 can be configured to collapse in a controlled manner, e.g., with at least a portion deflected away from inner wall of the sheath 28 when disposed in the sheath. As shown, the struts 19 can comprise knees 102, which can serve to space distal ends of the struts 19 (e.g., at or near the contact pads 24 or hooks) from the inner wall of the sheath 28, such that there is a space 46 between the contact pads 24 or hooks and the inner wall of the sheath 28 in the collapsed configuration within the sheath 28.

The knees 102 can be of the same configuration for each of the struts 19 in one embodiment. In such an embodiment, the struts 19 may all collapse or fold in the same manner within the sheath 28. In another embodiment the knee 102 of one or more struts 19 can be differentiated from the knee 102 of one or more other struts 19 such that the struts are collapsed or folded in different manners. As explained herein, in various embodiments, the struts can be longitudinally-aligned or longitudinally-offset or staggered. For example, a pair of opposing struts 19 (e.g., disposed radially opposite one another) can have knees 102 that cause the opposing strut of the pair to collapse prior to the collapsing of other struts 19 of the pump 2. In one example, the pump 2 has four struts 19. Two opposing struts 19 are configured to bend at the knees 102 prior to the bending of the knees of the other struts 19. As such, the two opposing struts 19 can be collapsed to a position between the other two struts to provide a compact arrangement. The knees 102 can be configured such that some struts undergo a greater degree of bending or collapsing. Thus, the space 46 between the contact pads 26 and the inner wall of the sheath 28 can be two to six (and in some cases three to four) times greater for one or more, e.g., a pair of, struts than for one or more, e.g., another pair of struts 19, which can be provided to avoid tangling of the struts. Accordingly, in various embodiments, some struts may be structured to collapse first when engaged with the sheath 28, and the remaining struts can collapse as the sheath 28 induces the collapsing of the initial struts.

In some embodiments, one or more struts comprises knees 102 that can control the order of collapsing of the struts. For example, one or more struts can have a knee 102 positioned more proximally compared to the position of the knees 102 of one or more other struts. In one example, two opposing struts 19 can have knees 102 disposed more proximally than are the knees 102 of another strut 19. In one example, a first set of opposing struts 19 have knees 102 disposed more proximally than a second set of struts 19 disposed approximately 90 degrees offset from the first set of struts 19. This can allow the first set of struts to be more completely folded by distal advancement of the sheath 28 before a more complete folding of the second set of struts 19. In a further variation, knees 102 can be longitudinally spaced apart on adjacent struts 19 so that adjacent struts fold at different times or rates. The illustrated embodiments includes the knees 102, but in other embodiments, no knees may be provided. For example, the struts 19 can be retracted at different rates by hinges and/or by modifying material thickness or properties in or along the length of one or more struts 19 to control the timing or rate of folding upon advancing the sheath 28. A living hinge structure can be formed along the length of one or more struts 19 to control timing, rate, and/or sequence of retraction of the struts 19. In one example, an area of reduced thickness transverse to the length of a strut 19 causes the strut to fold or bend when a sheath is advanced across the reduced thickness area. By offsetting the longitudinal position of reduced thickness areas in the struts 19, the sequence of retraction can be controlled.

In the collapsed configuration, the struts 19 can be compressed to a diameter or major lateral dimension at one or more locations that is approximately the same as (or slightly smaller than) the diameter of the shroud 16. Thus, as shown in the collapsed configuration of FIG. 1H, at least a portion of the struts 19 are compressed to a diameter or major lateral dimension that is smaller than the major lateral dimension or diameter of the pump housing 35, shroud 16 and/or the drive unit 9. In some embodiments, at least a portion of the struts has a major lateral dimension that is no more than a major lateral dimension of the pump housing 35. In some embodiments, at least a portion of the struts has a major lateral dimension that is less than a major lateral dimension of the pump housing 35 and/or the motor housing 29. The patient can be prepared for the procedure in a catheterization lab in a standard fashion, and the femoral artery can be accessed percutaneously or by a surgical approach. The sheath 28 (or a dilator structure within the sheath 28) can be passed over a guidewire and placed into the treatment location, for example, in the descending aorta. After the sheath 28 is placed (and the dilator removed), the pump 2 can be advanced into the sheath 28, with the pump 2 disposed in the mid-thoracic aorta, approximately 4 cm below the take-off of the left subclavian artery. In other embodiments, the pump 2 and sheath 28 can be advanced together to the treatment location. Positioning the pump 2 at this location can beneficially enable sufficient cardiac support as well as increased perfusion of other organs such as the kidneys. Once at the treatment location, relative motion can be provided between the sheath 28 and the pump 2 (e.g., the sheath 28 can be retracted relative to the pump 2, or the pump 2 can be advanced out of the sheath 28). The struts 19 of the localization system can self-expand radially outwardly along the radial axis R due to stored strain energy into the deployed and expanded configuration shown in FIGS. 1A-1C. In some embodiments, such as those in which the vasculature is accessed by the femoral artery, the struts 19 can extend distally, e.g., distally beyond a distal end of the shroud 16 and/or the impeller 6. In other embodiments, as explained herein, the pump 2 can be delivered percutaneously through a subclavian artery. In such embodiments, the struts 19 may extend proximally, e.g., proximal the pump housing 35 and/or the motor housing 29. In still other embodiments, multiple pluralities of struts may extend proximally and distally relative to the pump 2. The convex contact pads 24 can engage the blood vessel wall to stabilize (e.g., assist in anchoring) the pump 2 in the patient's vascular system. Once at the treatment location, the clinician can engage the control system to activate the motor 30 to rotate the impeller assembly 4 to pump blood.

Thus, in some embodiments, the pump 2 can be inserted into the femoral artery and advanced to the desired treatment location in the descending aorta. In such arrangements, the pump 2 can be positioned such that the distal end 22 is upstream of the impeller 6, e.g., such that the distally-located first fluid port 27 is upstream of the second fluid port(s) 25. In embodiments that access the treatment location surgically or percutaneously via the femoral artery, for example, the first fluid port 27 can serve as the inlet to the pump 2, and the second ports 25 can serve as the outlet(s) of the pump 2. The struts 19 can extend distally beyond a distal end of the pump housing 35. In other embodiments, however, the pump 2 can be inserted percutaneously through the left subclavian artery and advanced to the desired treatment location in the descending aorta. In such arrangements, the pump 2 can be positioned such that the distal end 22 of the system 1 is downstream of the impeller 6, e.g., such that the distally-located first fluid port 27 is downstream of the second fluid port(s) 25. In embodiments that access the treatment location through the left subclavian artery, the second fluid port(s) 25 can serve as the inlet(s) to the pump 2, and the first port 27 can serve as the outlet of the pump 2.

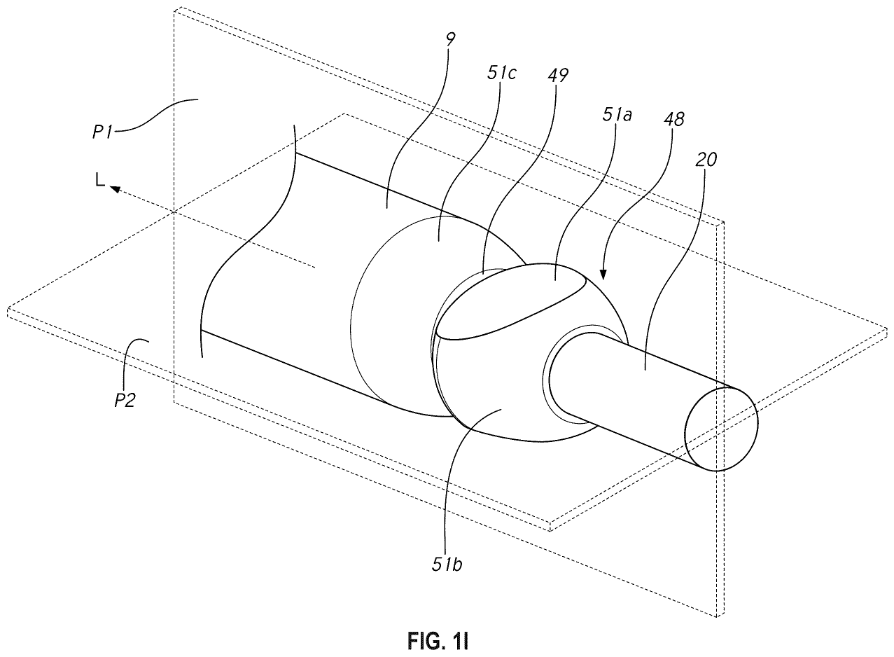

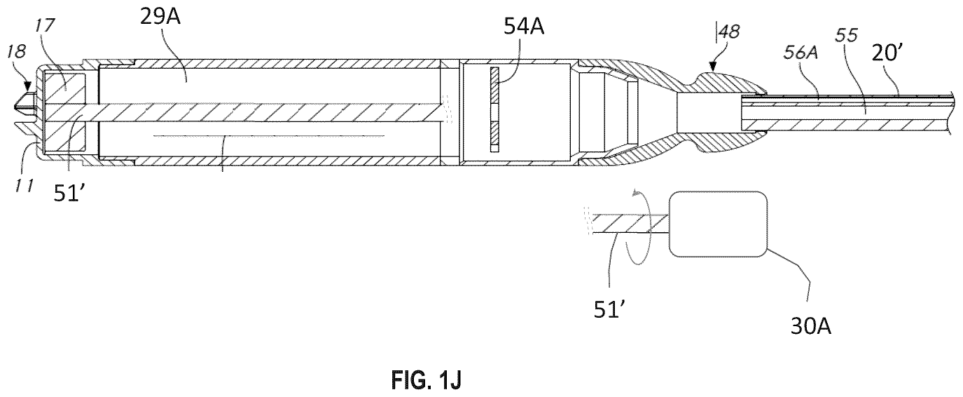

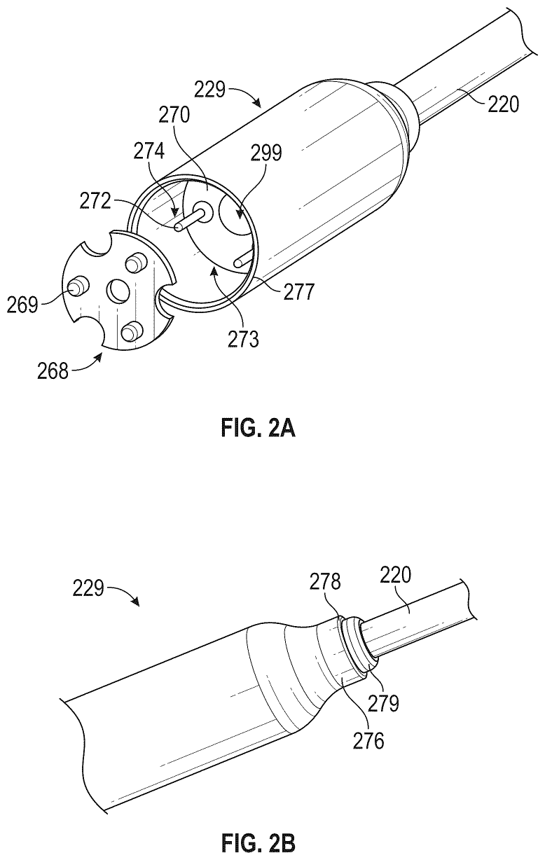

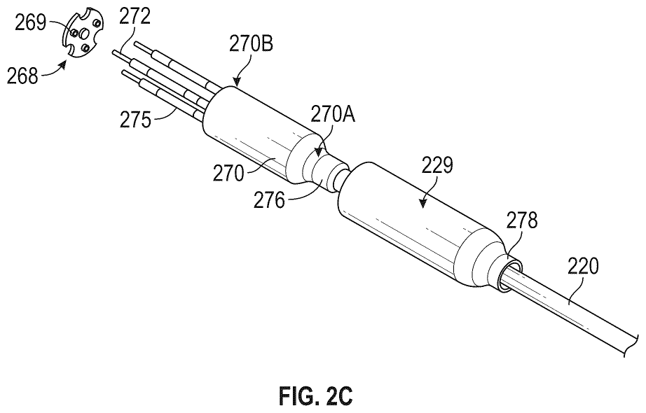

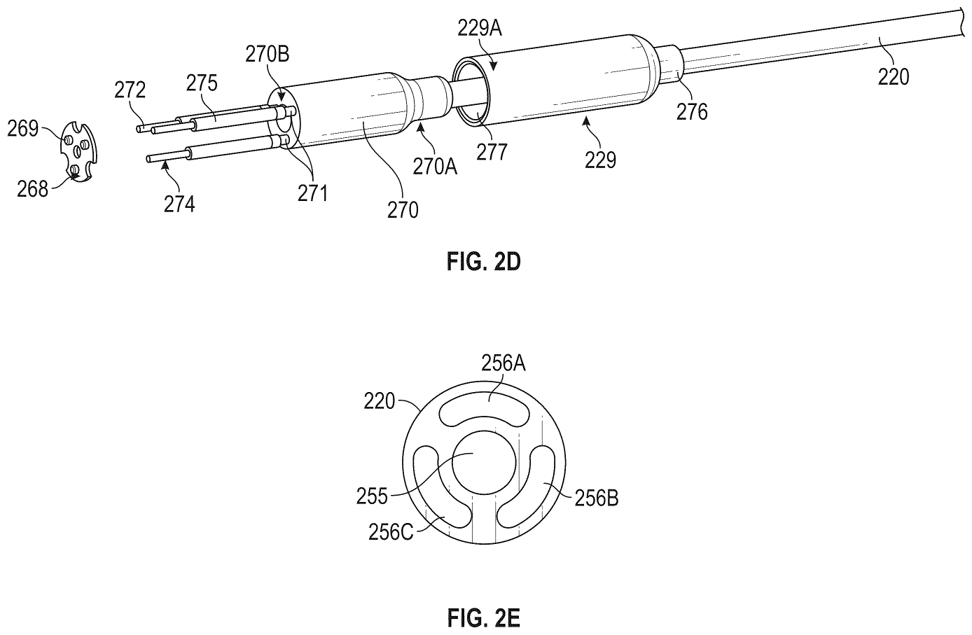

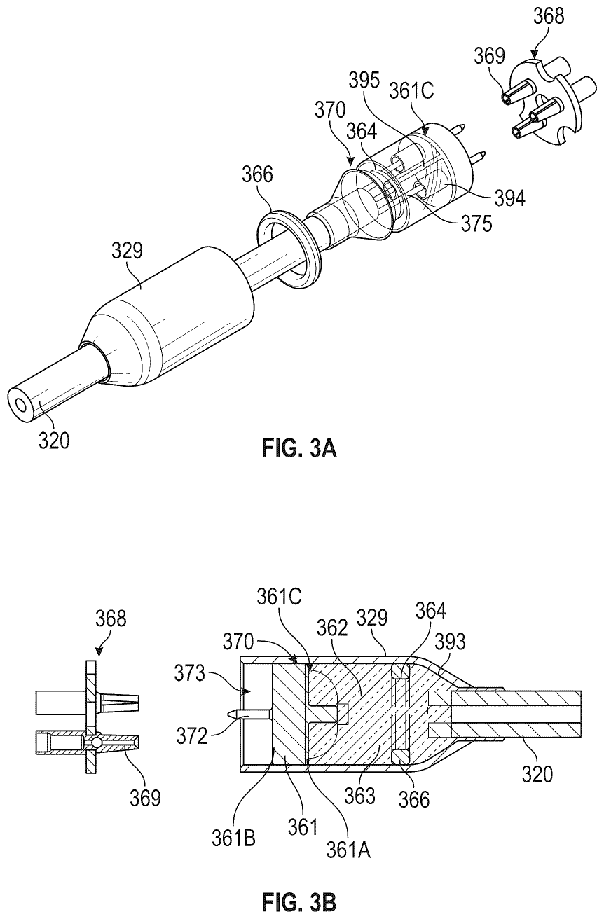

When the treatment procedure is complete, the pump 2 can be removed from the patient. For example, in some embodiments, the pump can be withdrawn proximally (and/or the sheath 28 can be advanced distally) such that a distal edge of the sheath 28 engages with a radially-outer facing surface 43 of the struts 19. In some embodiments, the distal edge of the sheath 28 can engage with the knees 102 of the struts (see, e.g., FIGS. 2A-3C). The distal edge of the sheath 28 can impart radially-inward forces to the radially-outer facing surface 43 (e.g., at approximately the location of the knees 102) to cause the struts 19 to collapse and be drawn inside the sheath 28. Relative motion opposite to that used for deploying the pump 2 can be provided between the sheath 28 and the pump 2 (e.g., between the sheath 28 and the impeller assembly 4 and pump housing 35) to collapse the struts 19 into the sheath 28 in the collapsed configuration. In some embodiments, the pump 2 can be withdrawn from the sheath 28 with the sheath 28 in the patient's body, and the sheath 28 can be subsequently used for another procedure or removed. In other embodiments, the sheath 28 and the pump 2 can be removed together from the patient's body.

The foregoing description includes embodiments in which a proximal end of a drive shaft 51 is located in the drive unit 9. The proximal end of the drive shaft 51 and the motor 30 are disposed within the body in use. FIG. 1J shows another embodiment in which a motor 30A is disposed outside the body in use. An elongate, flexible shaft 51′ is coupled at a distal end with the drive magnet 17. The shaft 51′ extends through an elongate body 20′ and is or can be coupled at a proximal end thereof with a motor 30A. The motor 30A can be larger than the motor 30 since it need not be disposed within the profile of the sheath 28. The elongate body 20′ may have one or more lumens. The shaft 51′ may extend through the central lumen 55. One or more outer lumens 56a may be provided to flow a fluid into the system to lubricate and/or cool the shaft 51′. Rotation of the proximal end of the shaft 51′ by the motor 30a results in rotation of the entire length of the shaft 51′ through the elongate body 20′ and also results in rotation of the drive magnet 17. Rotation of the drive magnet 17 causes rotation of one or more magnets in the impeller 6 to create flow through the pump 2 by virtue of magnetic attraction of these magnets across the distal drive unit cover. In other embodiments, the shaft 51′ can be directly mechanically coupled to the impeller 6 such that rotation does not depend on magnetic coupling. One or more shaft rotation supports 54A can be provided within a distal housing 29A to support a distal portion of the shaft 51′. The elongate body 20′ and/or the shaft 51′ can comprise a tether to control or to aid in control of the position of the pump, e.g., to counter thrust forces of the impeller 6 to reduce or minimize movement of the pump 2 in operation.

Additional details of the pump 2 and related components shown in FIGS. 1A-1H may be found throughout International Patent Application No. PCT/US2020/062928, filed on Dec. 2, 2020, the entire contents of which are incorporated by reference herein in their entirety and for all purposes.

Prior work is detailed in U.S. Pat. No. 8,012,079 and U.S. Pat. Pub. No. 2017/0087288, which are both fully incorporated by reference herein.

### II. FEEDTHROUGH ASSEMBLIES

Implanted electromechanical devices utilize the transmission of electrical power. The drivelines or power leads that transmit this electrical power are made from biocompatible materials that remain stable for the duration of the implant or therapy. Certain parts of the electromechanical device may use non-biocompatible materials, which are often sealed off from the biological environment. Additionally, the conductors are typically electrically isolated from one another and from the device casing. Some devices may also allow manipulation via the driveline, such as applying tension, support (compression), or both. For example, as shown in FIGS. 1D-1F, an elongate member (e.g., power lead 20) can extend from the treatment location to outside the body of the patient, and can provide electrical power (e.g., electrical current) and/or control to the motor 30. In other embodiments, a spinning drive shaft can be provided through the elongate member. The devices, systems, and methods disclosed herein can be utilized in combination with any of the devices, systems, and methods disclosed in U.S. patent application Ser. No. _________________, having attorney docket number PRCY. 029A, filed Nov. 7, 2025, entitled “MODULAR STRUT ASSEMBLY FOR INTRAVASCULAR DEVICES,” and in U.S. Patent Application No. _________________, having attorney docket number PRCY. 031A, filed Nov. 7, 2025, entitled “MODULAR ASSEMBLY OF IMPLANTABLE ELECTROMOTIVE MICROMECHANCIAL BLOOD PUMP,” the entire contents of each of which are hereby incorporated by reference herein in their entirety and for all purposes.

In the arrangement of FIGS. 1A-1J, the power lead 20 includes an elongate member comprising a thermoplastic polyurethane multi-lumen extrusion, which contains a plurality of (e.g., three) conductor lumens 56A-56C and a central lumen 55. The conductor lumens 56A-56C house electrical cables (e.g., MP35N silver alloy cable bundles), while the central lumen 55 is left empty and can be used to support the pump during deployment with a guidewire. During retrieval, the central lumen 55 can accommodate an element that provides positional support (e.g., both tension and compression) and stiffness and/or support to a catheter assembly that traverses the power lead 20. The connector end of the device is shown in FIGS. 1A and 1G. For example, there are a plurality of (e.g., three) electrical contacts 58A-58C (e.g., ring contacts) on the connector end, along with a retrieval feature 48 for latching and retrieving the pump at the proximal end of the driveline. The ring contacts 58A-58C can be made of a platinum/iridium alloy for excellent corrosion resistance. The elongate member 20 (e.g., polyurethane extrusion) and electrical cable bundles are designed to provide excellent flexibility while maintaining relatively high tensile strength. The cable assembly is terminated with a crimp (e.g., gold crimp sleeves) at the distal end of the driveline (e.g., at distal ends of the cables). During pump assembly, these crimp sleeves are inserted into the holes on the drive unit printed circuit board (PCB) and soldered in place, which can provide the primary tensile strength of the assembly.

In the arrangements described above, a first portion of the motor housing 29 is advanced to mate with the housing for the drive unit 9, and the cavity 229A is filled with potting compound. The potting compound enhances the tensile strength of the assembly, provides electrical isolation between components, and provides protection against electrical leakage through the housing. Once the potting compound cures, a second component of the housing is filled with a different potting or sealing material and is advanced to mate with the first component. After curing, the connection forms a fluid seal, preventing intrusion along the power lead into the housings. The housings are laser welded to each other and to the main pump enclosure. This multi-stage, potting specific design provides a slow manufacturing process, complexity in qualifying the process, and potential unsuitability for long-term implantation, as the potting material may degrade over time, allowing ionic fluid ingress.

Various embodiments disclosed herein incorporate a pin-terminated feedthrough to the distal end of the power lead. In this embodiment, the existing crimp sleeves can be replaced by pins that protrude through a ceramic and metal feedthrough assembly. The circuit board (e.g., printed circuit board or PCB) on the drive unit can be configured with mating sockets, eliminating both the soldering and potting steps required for pump assembly, which yields several advantages over the above-described implementation. For example, the embodiments disclosed herein can significantly simplify the final assembly of the pump. Moreover, hermeticity of the pump can be quantitatively assessed by a helium leak test (as opposed to the implementations above, which, while fluid-tight, may not be helium-tight). Qualification of the procedure can also be simplified, as process qualification at the high-value step (e.g., at the assembled pump stage) can be limited to ensuring quality of the laser welds. The ceramic-to-driveline side of the feedthrough can use a potting compound that can be flowed in, ensuring that the cavity is fully filled. This allows the use of an industry-standard material known to prevent ionic fluid intrusion for extended therapy durations. Moreover, the embodiments disclosed herein can be keyed for blind assembly, allowing the removal of volumes required for digital manipulation of the existing crimp sleeves, thus reducing overall size and length.

FIGS. 2A-2E illustrate an example embodiment of a housing 229 for electrical pins 274 configured to electrically connect to a therapy device, which can be an electromechanical device. The housing 229 can be connected to an elongate body 220 (which can be similar to or the same as the power lead 20 above). The elongate body 220 can have a proximal end and a distal end configured to electrically and mechanically connect to a therapy device such as a blood pump as explained above. Although the illustrated embodiments depict a blood pump, it should be appreciated that the power leads and devices described herein can be used with any suitable therapy device, such as any suitable medical device (e.g., a medical device sized to fit within the human body). The therapy device (e.g., pump) can be disposed adjacent to the distal end of the power lead 229 and can be connected to a controller at the proximal end of the power lead 229. The elongate body 220 can comprise a plurality of lumens 256A-256C and a central lumen 255. Each lumen 256A-256C can be configured to house a portion of a length of a conductor of a plurality of conductors. The conductors can be electrical wires or cables configured to provide electrical power to a motor in some embodiments. In other embodiments at least one rotating drive cable that directly drives a rotating or movable component at the distal portion of the device can be disposed in one of the lumens of the elongate body 220.

The housing 229 can enclose a feedthrough body 270 having a proximal end 270A and a distal end 270B and comprising a plurality of channels 271. Each channel 271 can be configured to house a proximal portion of a connector pin 272 configured to be electrically connected to a conductor 275 of the plurality of conductors 275. The housing 229 can have a length greater than the length of the feedthrough body 270 such that a distal portion 274 of the connector pin 272 is cantilevered from the feedthrough body 270. The distal portion 274 of the connector pin 272 can be disposed in a blind recess 273 extending from an opening of the housing 229 to the distal end 270B of the feedthrough body 270. Once assembled, the distal portion 274 of the connector pin 272 can be disposed in a cavity defined at least in part by the housing 229 and a distal portion of the housing 229 that forms the cavity.

In various embodiments, the feedthrough body 270 comprises a ceramic material. Example of such materials can include alumina oxide (AL203). In various embodiments, the body of the housing 229 can comprise an inert metal. In some embodiments, the body of the housing 229 can comprise titanium (commercially pure or an alloy). As shown in, e.g., FIGS. 2B-2D, a welding feature 276 can be disposed between the proximal end 270A of the feedthrough body 270 and the elongate body 220. The welding feature 276 (also referred to as a weld purchase component) can comprise a material configured to enhance a weld purchase to the housing 229 (e.g., both the feature 276 and the housing 229 can be titanium in some implementations). As shown, the housing 229 can include openings at each of an enlarged distal end 277 and a tapered proximal end 278, the opening at the distal end 277 configured to receive a feedthrough body assembly, a proximal end 279 of the feedthrough body assembly configured to protrude through the opening at the proximal end 278 of the housing 229. In some embodiments, the welding feature 276 of the feedthrough body assembly comprises a short cylindrical member that can protrude through an opening in the proximal end 278 when the feedthrough body 270 is advanced fully into the housing 229. The cylindrical member is exposed at the proximal end 278 to enhance a weld between the housing 229 and an assembly including the feedthrough body 270 and the welding feature 276.

In various embodiments, the feedthrough body 270 comprises a sealing passage 299 configured to convey a sealant into cavities in and/or around the feedthrough body 270. By this process, a sealant can be disposed in interstices of the power lead in and around the feedthrough body 270. Beneficially, the sealant and sealing passage can prevent fluids (e.g., blood or interstitial fluid in the patient's body) from contaminating electrical and/or mechanical components of the assembly.

The therapy device (e.g., a blood pump in some embodiments), can include an electrical circuit (e.g., formed in a circuit board 268) electrically connected to a plurality of sockets 269. Each socket 269 of the plurality of sockets 269 can be configured to receive one of the connector pins 272 of the plurality of connector pins 272 to electrically connect the therapy device to the controller. Beneficially, the sockets 269 can comprise a snap-fit connection or press fit that provides a mechanical and electrical interconnection upon insertion and/or snapping of the pins 272 into the sockets 269, which can obviate the use of solder or other conductive adhesives.

In some embodiments, a weld can be formed between the distal end 277 of the housing 229 of the power lead and a surface of the therapy device adjacent to the plurality of sockets 269 so as to mechanically connect the housing 229 to the remainder of the therapy device (e.g., the pump). Beneficially, the interconnection of components can be done in a hermetic manner that can provide better than a liquid seal, e.g., the seal can be sufficiently strong so as to prevent leaking of helium in a helium leak test.

FIGS. 3A-3B illustrate additional embodiments of a feedthrough assembly for a therapy device, such as an electromechanical device (e.g., a heart pump). Unless, otherwise noted, components of FIGS. 3A-3B may be the same as or generally similar to like numbered components of FIGS. 2A-2E, with reference numerals incremented by 100 relative to FIGS. 2A-2E. Unlike FIGS. 2A-2E, in FIGS. 3A-3E, the feedthrough body 370 can comprise a disk 361 disposed in the housing 329. The disk 361 is relatively short, e.g., occupying only a fraction of the housing 329. The disk 361 can occupy a portion of but less than half of the axial length of the housing 329, less than a quarter of the axial length of the housing 329, less than twenty percent of the axial length of the housing 329. A distal portion 361B of the disk can at least partially define a portion of the blind recess 373, and a proximal portion 361A of the disk can be disposed distal to a volume 362 through which each of the conductors of the plurality of conductors 375 can be routed. As shown, an enclosure 363 can be disposed between the proximal end 361A of the disk 361 and the distal end 361B of the elongate body 320. The enclosure 363 can be configured to at least partially surround the volume 362 through which each of the conductors 375 of the plurality of conductors 375 can be routed. In various embodiment, the enclosure 363 can comprise a polymeric body, such as a molding or potting compound.

As shown, the enclosure 363 can include a groove 364 configured to receive a seal member 366, such as an O-ring. The enclosure 363 can be disposed within the housing 329 as shown. In FIG. 3B, an adhesive 393 can be disposed between an outer surface of the enclosure 363 and an inner surface of the housing 363. The adhesive 393 can be disposed within the enclosure 363 to control fluid ingress into the elongate body 320. For example, the volume 362 can be at least partially occupied by the adhesive 393 to control fluid ingress into the elongate body 320.

In FIGS. 3A-3B, a conductor passage assembly can include a support element 361C (e.g., part of the disk 361 or a separate thin plate disposed on the proximal portion 361A) and a plurality of tubular bodies 394. Each tubular body 394 can be configured to receive a distal portion of a conductor 375 of the plurality of conductors 375. The support element 361C can comprise a planar portion (e.g., a thin plate-like element coupled with the proximal surface of the disk 361) disposed proximal of the feedthrough body and a flange 395 extending proximally from the planar portion between two of the tubular bodies 394 of the plurality of tubular bodies 394. In the illustrated embodiment, each adjacent pair of tubular bodies 394 can be separated by a flange 395 of the support element. The flanges 395 provide physical isolation between the tubular bodies 394 and a volume disposed between them can be filled with potting compound (e.g., the adhesive 393) to enhance the isolation.

In various embodiments, the housing 229, 339 can comprise a feature configured to be coupled with a motor housing enclosing a motor configured to be activated to produce rotational motion (e.g., to drive an impeller of a pump as explained herein). A connection portion can include a coupling surface and an electrical circuit (e.g., circuit board 268, 368), with the electrical circuit being electrically connected to the plurality of pin receptacles (e.g., sockets 269, 369), each pin receptacle or socket 269, 369 can be configured to receive a pin of a plurality of pins 272, 372 of the power lead to electrically connect the motor to the controller. The coupling surface is configured to facilitate a sealing connection to the power lead, e.g., by way of a circumferential weld. In some embodiments, the pin receptable or socket 269, 369 can be configured to electrically connect to a pin 272, 373 of the power lead via a press-fit connection.

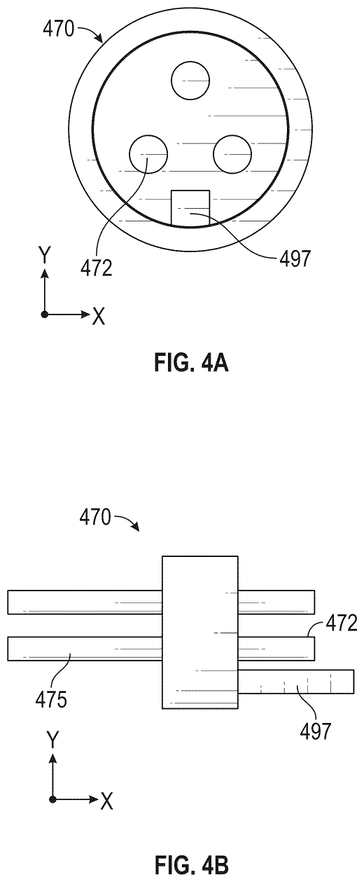

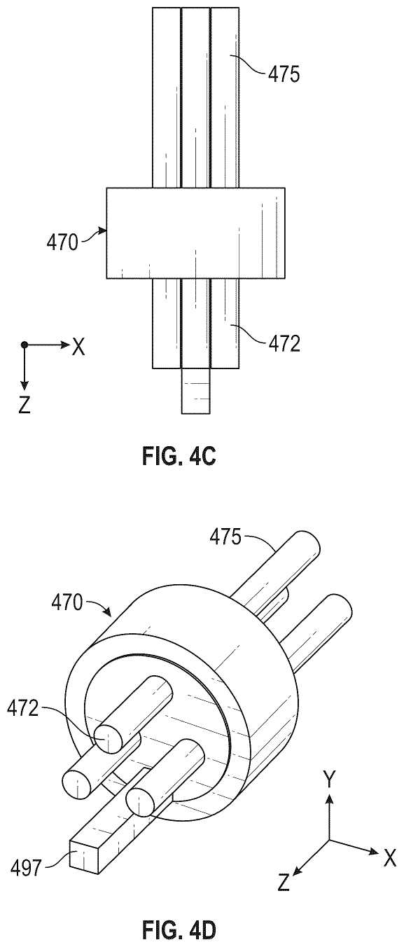

FIGS. 4A-4D illustrate an embodiment of a feedthrough body 470 including an alignment key 497 to facilitate automatic alignment with the remainder of the pump. Unless, otherwise noted, components of FIGS. 4A-4D may be the same as or generally similar to like numbered components of FIGS. 3A-3B, with reference numerals incremented by 100 relative to FIGS. 3A-3B. In the embodiment of FIGS. 4A-4D, one or more alignment keys 497 can be provided to extend from the feedthrough body 470 generally parallel to the pins 472. The keys 497 can be provided for plugging in the pins 472 into the motor unit, e.g., through the circuit board 468 to align the pins 472 with the remainder of the motor and pump unit. Beneficially, the key 497 can be provided asymmetrically to ensure that the correct pins 472 are connected to the correct connectors on the circuit board 468. Without the key 497, the symmetry of the pins can make it difficult to identify the correct electrical connections during assembly. The key 497 can engage with a corresponding groove or recess in the motor body and/or the circuit board 468. In various embodiments, the alignment key 497 can be longer than the pins 472, e.g., the key 497 can extend farther distally than the distal ends of the pins 472. An additional advantage of the key 497 arises when the key 497 is longer than the pins 472, in that the key engages the corresponding groove or recess before the pins/sockets are engaged. As such, an assembler is prevented from misaligning the pins with the sockets which can result in damage to the pins or sockets. This will permit the assembly to be assembled “blind” (e.g., without directly visualizing the pins/sockets) which can also shorten the assembly time. The key 497 can be coupled with either be on the pin or the receptacle side.

The driveline assembly with integrated feedthrough and housings disclosed herein in FIGS. 2A-4D can significantly reduce manufacturing complexity of the finished electromechanical device. Hermeticity can be quantitatively verified at the subassembly level using a helium leak rate test. Final assembly can then be expedited, e.g., by only plugging in of the assembly and a single laser weld. This design ensures that the wet side of the feedthrough remains free of ionic fluids, as used for permanent implants. Furthermore, this design permits the use of a central lumen to enable manipulation of the device through the driveline (e.g., tension, compression, or both).

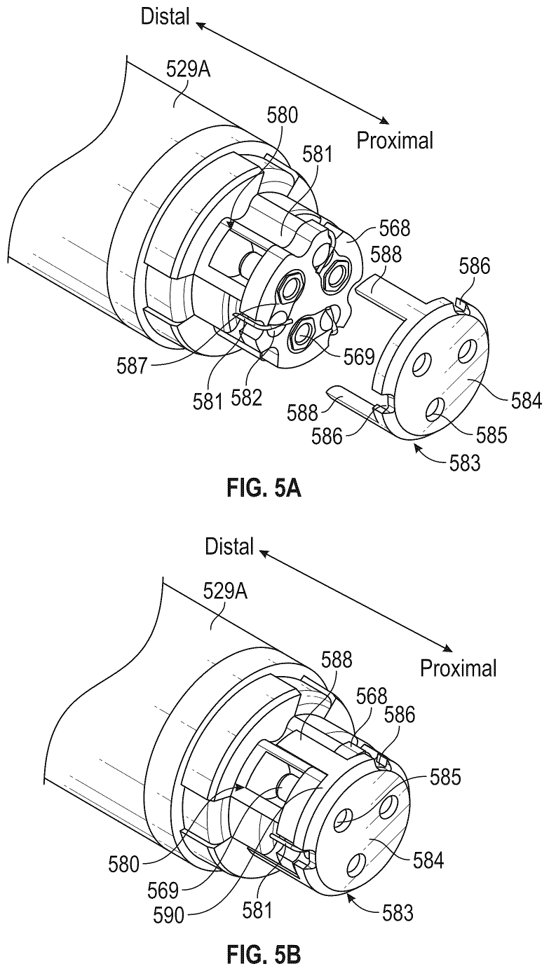

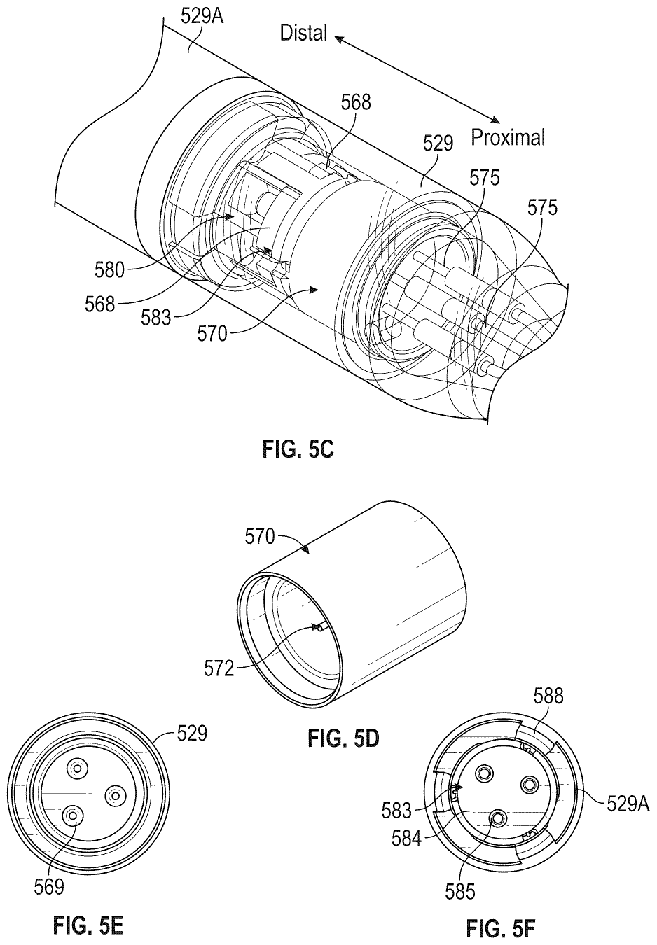

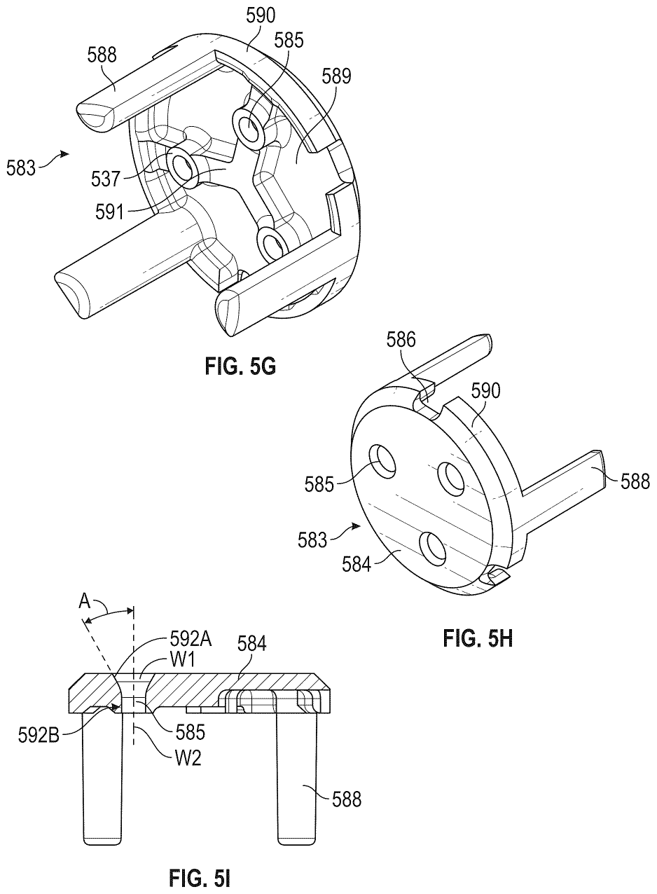

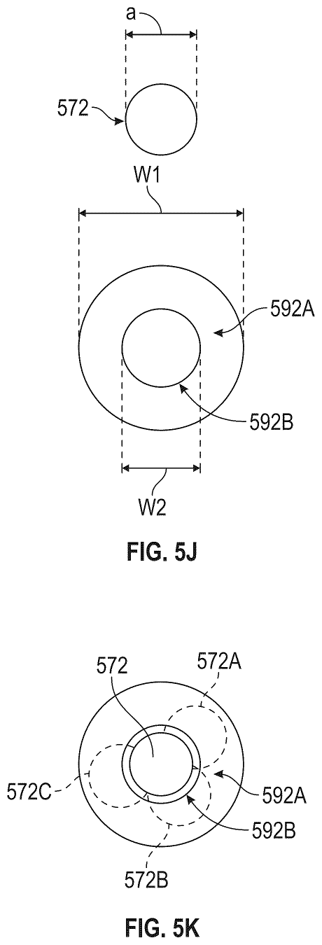

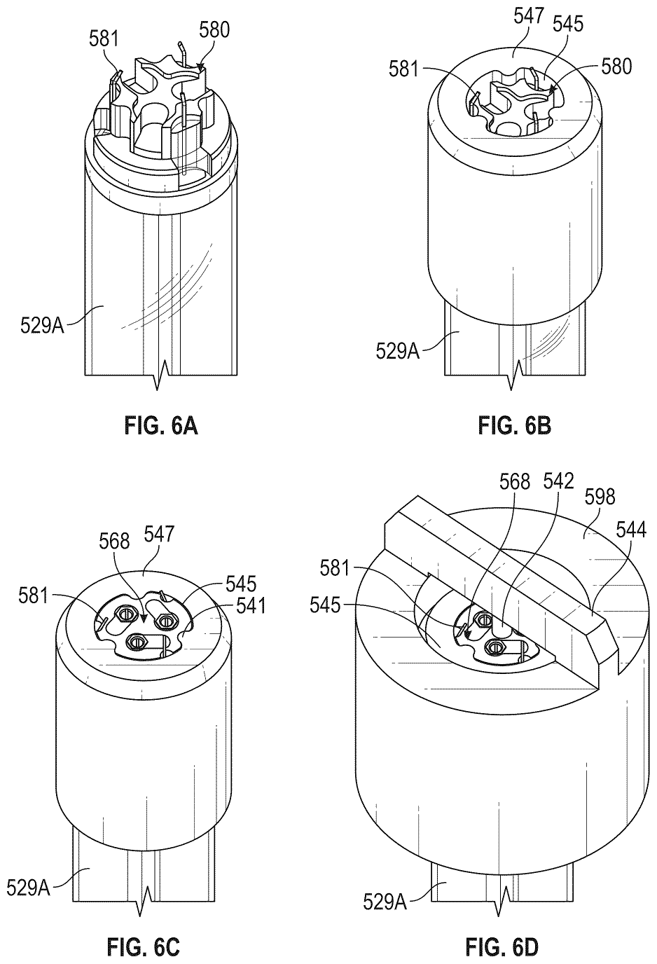

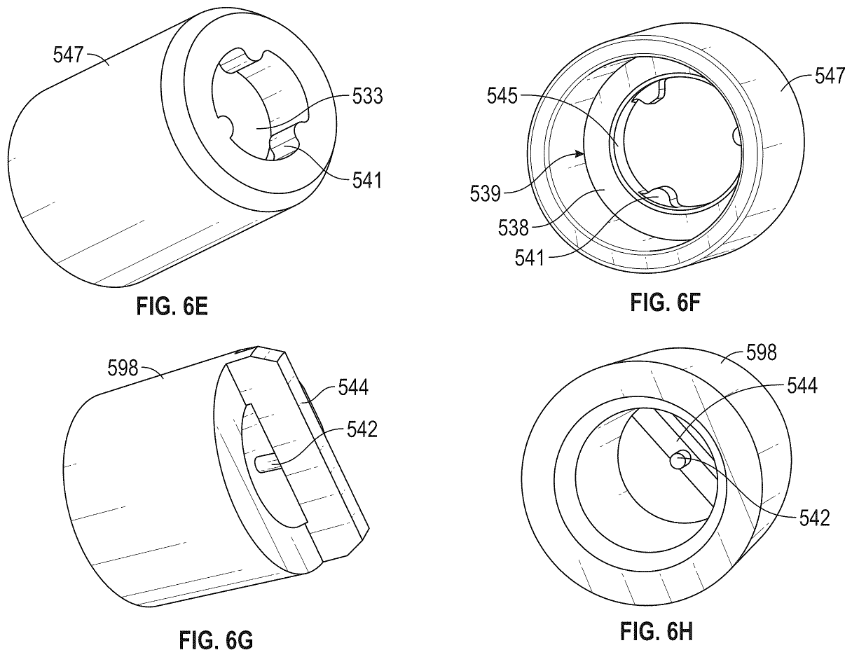

FIGS. 5A-6H illustrate additional examples of a feedthrough assembly for a therapy device, such as an electromechanical device (e.g., a heart pump). Unless, otherwise noted, components of FIGS. 5A-6H may be the same as or generally similar to like numbered components of FIGS. 2A-4D, with reference numerals incremented by 100 relative to FIGS. 4A-4D. During assembly, it can be challenging to align the feedthrough or connector pins (e.g., connector pins 272, 372, 472) with the sockets (e.g., sockets 269, 369, 469) that receive the pins. For example, in some embodiments, the connector pins can be brazed into a ceramic material and may essentially float for some time in a liquid (e.g., liquid gold), which can create a zone of uncertainty in the position of the connector pins (e.g., the radial and/or circumferential position of the pins). Beneficially, various embodiments disclosed herein can accommodate the positional tolerances of the connector pins while protecting the connection between motor windings and the solder pad on the circuit board. The embodiments disclosed herein can also provide a tactile feedback on alignment between the connector pins and the sockets during blind assembly, and can further provide alignment between the circuit board (e.g., circuit board 268, 368, 468) and the motor unit.

FIGS. 5A-5C schematically illustrate a portion of a feedthrough that facilitates connection of connector pins 572 (see FIG. 5D) (or pins 272, 372, 472) with sockets 569 of the circuit board 568. The circuit board 568 and sockets 569 may be the same as or generally similar to the circuit board 268, 368, 468 and sockets 269, 369, 469 of FIGS. 2A-4D. Accordingly, the circuit board 568 can be positioned within the housing 529 (which may be similar to or the same as housing 229, 329, 429), and longitudinally between the feedthrough body 570 (which can be the same as or generally similar to feedthrough bodies 270, 370, 470) and the motor and motor housing 529A (also shown as motor 30 and motor housing 29 in FIGS. 1C-1D). Thus, in FIGS. 5A-5C, the feedthrough body 570 can be positioned proximal the circuit board 568, which in turn can be proximal the motor housing 529A.

As explained above, it can be challenging to align the connector pins 572 extending from the feedthrough body 570 (FIG. 5D) with the sockets 569 in the circuit board 568. Accordingly, in various embodiments, a pin guide 583 can be provided to assist in aligning the connector pins 572 with the sockets 569 in the circuit board 568. FIG. 5E illustrates a proximally-facing plan view of the sockets 569, while FIG. 5F illustrates a distally-facing plan view of the pin guide 583. FIGS. 5G and 5H are schematic perspective view of the pin guide 583, and FIG. 5I is a schematic side sectional view of the pin guide 583. The pin guide 583 can be formed of an electrically insulating material, e.g., a polymer, so as to provide electrical separation between connector pins 572. In various embodiments, the pin guide 583 can comprise a seamless, unitary structure, for example, a molded structure.

The pin guide 583 can comprise a base member 584 (which can be rounded or circular), a rim 590 disposed circumferentially about the base member 584 and extending non-parallel (e.g., transverse to) the base member 584 (for example, extending distally from the base member 584). One or more fingers 588 can extend non-parallel (e.g., transverse) relative to the base member 584. The fingers 588 can be longer than the rim 590 along the longitudinal direction. The fingers 588 can be disposed at an outer periphery of the base member 584. In the illustrated embodiment, the finger(s) 588 can extend from the rim 590 in a direction non-parallel relative to the base member 584. In the illustrated embodiment, the pin guide 583 includes a plurality of (e.g., three) fingers 588, but it should be appreciated than any suitable number of fingers 588 can be provided. For example, the pin guide 583 can include one, two, four, or more than four fingers 588. In the illustrated embodiment, the fingers 588 are disposed approximately equidistantly about the circumference of the base member 585 (e.g., spaced apart by 120°). The base member 584 can comprise a plurality of pin holes 585 disposed about a central region of the base member 584. The connector pins 572 can be inserted through the pin holes 584 to connect to the sockets 569 of the circuit board 568. Although three pin holes 585 are shown in FIGS. 5A-5B, and 5F-5H, it should be appreciated than any number of pin holes 585 can be provided. For example, the base member 584 can comprise the same number of pin holes 585 as there are connector pins 572 to connect to the circuit board 568.

As shown in the exploded view of FIG. 5A, the circuit board 568 can be disposed on (e.g., adhered to) a proximal drive cap 580 (e.g., an end portion of the motor housing 529A) that is disposed at a proximal portion of the motor housing 529A. As shown in FIG. 5A, the drive cap 580 and the circuit board 568 can cooperate to define a channel 581 extending longitudinally along outer surfaces of the drive cap 580 and circuit board 568. As shown in FIG. 5B, the pin guide 583 can be connected to the drive cap 580 and the circuit board 568 by inserting the fingers 588 into and along the channels 581. In some embodiments, the pin guide 583 can be adhered to the circuit board 568 and/or drive cap 580. In other embodiments, the pin guide 583 can snap into or otherwise non-adhesively connect to the drive cap 580 and circuit board 568, e.g., by way of the fingers 588.

Further, as shown in FIGS. 5A-5B, the pin guide 583 can comprise a plurality of notches 586 in the rim 590. In various embodiments, the notches 586 can extend partially into the base member 584. As shown, each notch 586 can be disposed circumferentially between adjacent fingers 588. When assembled, as shown in FIG. 5B, the notches 586 can at least partially circumferentially overlap with motor wires 581 (which connect to the motor 30 to power and/or control operation of the motor 30) and solder connections 582 that connect the motor wires 581 with conductors of the circuit board 568. The notches 586 can physically accommodate the solder connections 582 and motor wires 581 such that the pin guide 583 does not interfere with the routing of the wires 581 between the motor and the circuit board 568. As shown in FIG. 5A, a conductive connecting portion 587 can electrically connect the solder connections 582 with the socket 569 that receives the connector pins 574.

As shown in FIG. 5G, a distal surface of the base member 584 can be proximally recessed relative to a distal end of the rim 590 such that a relief surface 589 is provided relative to distal surfaces 537 of the pin holes 585. For example, the relief surface 589 can be proximally recessed relative to the distal surface 537 of the pin hole 585. A web 591 can mechanically connect the pin holes 585 to one another. When assembled, distal surfaces 537 of the pin holes 585 may abut or contact proximal surfaces of the sockets 569, which may provide a longitudinal standoff between the relief surface 589 and the solder connections 582 and motor wires 581. In addition, as shown in FIG. 5I, the pin holes 585 can comprise a first tapered sidewall 592A and a second vertical sidewall 592A extending distally from the tapered sidewall 592A. The first tapered sidewall 592A can be tapered at an angle relative to a central axis Z of the pin hole 585 by an angle A. The angle A can be in a range of 10° to 50°, in a range of 20° to 40°, or in a range of 25° to 35°, e.g., about 30° in some embodiments. Beneficially, the tapered sidewall 592A can urge or guide the connector pins 572 from the wider tapered opening into the smaller diameter opening delimited by the second vertical sidewall. Accordingly, the pin hole 585 can have a first width W1 or diameter at a proximal surface of the base member 584 which can taper to a second width W2 or diameter that extends to the distal surface 537 of the pin hole 585. The second width W2 or diameter can be smaller than the first width W1 or diameter.

FIGS. 5J-5K illustrate an example of how the tapered pin hole 585 can facilitate alignment with the connector pin 572 due to the tolerances of the pins 572 so as to provide tolerance matching for the pins. As shown in FIG. 5J, and as explained above, the pin hole 585 can have the first width W1 at a proximal surface which can be larger than the second width W2 at the distal surface 537 of the pin hole 585. In various embodiments, the first width W1 can be in a range of 0.02 inches to 0.03 inches, in a range of 0.015 inches to 0.035 inches, or in any other suitable range. The second width W2 can be in a range of 0.01 inches to 0.02 inches, in a range of 0.005 inches to 0.025 inches, or in any other suitable range. In various embodiments, the first width W1 can be at least 1.3 times larger than the second width W2, at least 1.5 times larger than the second width W2, at least 1.75 times larger than the second width W2, or at least 2 times larger than the second width. Further, as shown in FIG. 5J, the connector pin 572 can have a width a that is smaller than the second width W2. For example, the second width W2 can be at least 1.005 times larger than the pin width a, at least 1.01 times larger than the pin width a, at least 1.025 times larger than the pin width a, at least 1.05 times larger than the pin width a, or at least 1.1 times larger than the pin width a.

As shown in FIG. 5K, by utilizing different widths W1, W2 for the proximal and distal surfaces and a tapered surface 592A, the pin hole 585 can guide the pin 572 into the lower or distal part of the hole 585 that is bounded by the vertical surface 592B. FIG. 5K illustrates three example initial contact locations between the pin 572 and the sloped surface 592A. For example, pin 572A illustrates a first contact location with the sloped surface 592A, pin 572B illustrates a second contact location with the sloped surface 592A, and pin 572C illustrates a third contact location with the sloped surface 592A, before the pin 572 is guided into the lower part of the hole 585 bounded by the vertical surface 592B with the second width W2.

Beneficially, the disclosed embodiments can facilitate alignment between the connector pins 572 and the sockets 569 to accommodate positional tolerances due to, e.g., fabrication of the pins 572 so as to provide tolerance matching for the pins. For example, the fingers 588 can serve to align the pin guide 583 to the circuit board 568 and drive cap 580, e.g., by way of the channels 581, which, in turn, can align the connector pins 572 extending through the feedthrough body 570 with the sockets 569. The tapered sidewall 592A of the pin holes 585 of the pin guide 583 can further assist in aligning the pins 572 with the vertical sidewall 592B and the sockets 569, at least because the larger width of the pin hole 585 at the tapered sidewall 592A can accommodate the positional tolerances of the pins 572. The tapered or ramped sidewall 592A can capture and guide the pins 572 into the smaller width opening delimited by the vertical sidewall 592B and into the opening of the socket 569. The pins 572 can accordingly extend through the pin holes 585 into the socket 569. The fingers 588 and channels 581 can cooperate to provide tactile feedback to the assembler during assembly of the system. Moreover, the pin guide 583 can include the relief surface 589 and notches 586 which can protect and/or accommodate the motor wires 581 and solder connections 582.

FIGS. 6A-6D schematically illustrate a process and components for attaching the circuit board 568 (or any of the circuit boards disclosed herein) to the drive cap 580. It can be challenging to support and align the circuit board 568 with the drive cap 580. In FIG. 6B, a support collar 547 can be provided around the drive cap 580 and motor wires 581. The support collar 547 can comprise an inner surface 545 shaped to conform to an outer surface of the circuit board 568. For example, the inner surface 545 can comprise a plurality of projections 541 (e.g., curved or semicircular) sized and shaped to extent into semicircular or curved notches or recesses in the circuit board 568. The collar 547 can provide a snug fit to prevent circumferential or lateral movement of the circuit board 568 during assembly. As shown in FIGS. 6E-6F, the collar 547 can comprise a lower cavity 539 and a shelf 538 at an upper end of the cavity 539. The shelf 538 can separate the lower cavity 539 from an upper cavity 533 into which the circuit board 568 is disposed during assembly. The shelf 538 can abut the drive cap 580 during assembly and the motor housing 529A can extend into the cavity 539.

As shown in FIGS. 6D, 6G, and 6H, an adhesive or glue can be applied to the drive cap 580 to adhere the circuit board 568 to the drive cap 580. To provide pressure to the circuit board 568 during the adhesive process, a compression tool 598 can be disposed around and over the collar 547 as shown in FIG. 6D. The compression tool 598 can comprise an arm 544 extending across a central opening of the tool 598 and include a longitudinally-projecting tip 542. The arm 544 can be integrally formed with an upper surface of an annular base. The compression tool 598 can be urged downwardly to press the tip 542 of the compression tool 598 against an upper surface of the circuit board 568. The tip 542 can accordingly maintain a position and/or orientation of the circuit board 568 as the circuit board 568 is adhered to the drive cap 580.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1” includes “1.” Phrases preceded by a term such as “substantially,” “generally,” and the like include the recited phrase and should be interpreted based on the circumstances (e.g., as much as reasonably possible under the circumstances). For example, “substantially spherical” includes “spherical.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y and at least one of Z to each be present.

Although certain embodiments and examples have been described herein, it should be emphasized that many variations and modifications may be made to the humeral head assembly shown and described in the present disclosure, the elements of which are to be understood as being differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, it will be understood by those skilled in the art that the scope of the inventions extends beyond the specifically disclosed embodiments to any and all embodiments having equivalent elements, modifications, omissions, combinations or sub-combinations of the specific features and aspects of the embodiments (e.g., of aspects across various embodiments), adaptations and/or alterations, and uses of the inventions as would be appreciated by those in the art based on the present disclosure. The limitations in the claims are to be interpreted fairly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

What is claimed is:

1. A power lead, comprising:
an elongate body having a proximal end and a distal end and being configured to electrically and mechanically connect to a therapy device adjacent to the distal end and to a controller adjacent to the proximal end, the elongate body comprising a plurality of lumens, each lumen configured to house a portion of a length of a conductor of a plurality of conductors;
a feedthrough body having a proximal end and a distal end and comprising a plurality of channels, each channel configured to house a proximal portion of a connector pin configured to be electrically connected to a conductor of the plurality of conductors; and
a housing enclosing the feedthrough body, the housing having a length greater than the length of the feedthrough body such that a distal portion of the connector pin is disposed in a blind recess extending between an opening of the housing and the distal end of the feedthrough body.

2. The power lead of claim 1, further comprising a welding feature disposed between the proximal end of the feedthrough body and the elongate body, the welding feature comprising a material configured to enhance a weld purchase to the housing.

3. The power lead of claim 1, wherein the feedthrough body comprises a sealing passage configured to convey a sealant into cavities in and/or around the feedthrough body.

4. The power lead of claim 1, further comprising an alignment key to facilitate automatic alignment with a portion of the therapy device.

5. An implantable device, comprising:
the power lead of claim 1; and
a therapy device comprising an electrical circuit electrically connected to a plurality of sockets, each socket of the plurality of sockets configured to receive one of the connector pins of the plurality of connector pins to electrically connect the therapy device to the controller.

6. The power lead of claim 1, wherein the feedthrough body comprises a disk disposed in the housing, a distal portion of the disk defining a portion of the blind recess, a proximal portion of the disk disposed distal to a volume through which each of the conductors of the plurality of conductors can be routed.

7. An implantable medical device comprising:
a first connector pin configured to electrically connect to a controller;
a therapy device comprising an electrical component connected to a first socket at an end portion of the electrical component, the first socket receiving the first connector pin; and
a pin guide connected to the end portion of the electrical component, the pin guide comprising a first pin hole aligned with the first socket, such that the first connector pin extends through the first pin hole to connect to the first socket.

8. The implantable medical device of claim 7, wherein the pin guide comprises one or more notches in a rim or outer periphery of a base member of the pin guide.

9. The implantable medical device of claim 8, wherein the electrical component comprises a motor housing and the end portion comprises a drive cap.

10. The implantable medical device of claim 9, further comprising a circuit board connected to the drive cap, the circuit board comprising the first socket.

11. The implantable medical device of claim 10, further comprising a motor wire electrically connected to the first socket by way of a solder connection.

12. The implantable medical device of claim 11, wherein a first notch of the one or more notches at least partially circumferentially overlaps the solder connection.

13. The implantable medical device of claim 7, wherein the first pin hole has a first width at a proximal surface and a second width at a distal surface, the second width smaller than the first width.

14. The implantable medical device of claim 7, wherein the first pin hole comprises a first tapered sidewall and a second vertical sidewall extending from the first tapered sidewall.

15. A pin guide comprising:
a base member;
a plurality of fingers extending non-parallel relative to an outer periphery of the base member; and
a first pin hole extending through the base member.

16. The pin guide of claim 15, further comprising a first notch in a rim of the pin guide or an outer periphery of the base member, the first notch disposed between two circumferentially-adjacent fingers.

17. The pin guide of claim 16, further comprising a plurality of notches including the first notch.

18. The pin guide of claim 15, wherein the first pin hole has a first width at a proximal surface and a second width at a distal surface, the second width smaller than the first width.

19. The pin guide of claim 15, wherein the first pin hole comprises a first tapered sidewall and a second vertical sidewall extending from the first tapered sidewall.

20. The pin guide of claim 15, further comprising a relief surface on a distal surface of the base member.
